(19) United States
(12) Patent Application Publication
(10) Pub. No.: US 2024/0003861 A1
(43) Pub. Date: Jan. 4, 2024

(54) AUTOMATED METHOD AND SYSTEM TO MEASURE RESIDUAL BIOCIDE IN SEAWATER

(71) Applicant: SAUDI ARABIAN OIL COMPANY, Dhahran (SA)

(72) Inventors: Fayez A. Al-Ammari, Abqaiq (SA); Osamah K. Al-Shurafa'a, Khurais (SA); Naif A. Al-Anazi, Abqaiq (SA)

(73) Assignee: SAUDI ARABIAN OIL COMPANY, Dhahran (SA)

(21) Appl. No.: 18/339,866

(22) Filed: Jun. 22, 2023

Related U.S. Application Data
(60) Provisional application No. 63/367,407, filed on Jun. 30, 2022.

(51) Int. Cl. G01N 33/18; G01N 35/00; C02F 1/50

(52) U.S. Cl. CPC G01N 33/18; G01N 35/00; C02F 1/50; C02F 2103/08

(57) ABSTRACT

A process and system for measuring biocide concentration in biocide treated seawater in an oilfield pipeline are provided. The system includes a seawater plant, a plurality of seawater sampling locations throughout the oilfield pipeline immediately downstream from the seawater plant, a surge tank, a water supply plant, a pH monitoring system, and an autosampler. Each of the plurality of seawater sampling locations has a pH monitoring system and an autosampler. The autosampler is used to collect a plurality of samples of the biocide treated seawater from the oilfield pipeline. The method includes measuring pH of the biocide treated seawater in the oilfield pipeline, when the pH of the biocide treated seawater is below 6.8, collecting a plurality of samples using an autosampler, correlating the pH of the biocide treated seawater with the biocide concentration, and stopping collection of the plurality of samples when the pH is 7.8 or higher.

### BACKGROUND

In oil exploration and production fields, seawater is pumped into strategically positioned injection wells to enhance the recovery of oil from the reservoir. The recovery of oil requires injection of water into oil-bearing reservoir rock in order to move the hydrocarbons to a production well where they can be produced to the surface. The length of the pipeline from the source of the water to the oil field where it is to be injected can be thousands of kilometers. The residence time of water in the pipelines can be significant and the likelihood of the presence of conditions that promote bacteria growth is high. The growth of bacteria in the pipeline can be prevented or greatly inhibited by the addition of a biocide at the water intake point that will have the effect of inhibiting bacterial growth throughout the pipeline.

The distribution pipelines normally form a grid to supply water to a number of injection wells in the vicinity of the production wells. Because of the overall length of the pipeline system, a drop in the effective concentration of biocide can occur at the point of use. The reduction in biocide concentration is due to the degradation of the active ingredient(s) present in the biocide formulation. Hence, it is important to know the actual concentration of biocide present in water at the point of use.

Many commonly used industrial water treatment biocide formulations contain formaldehyde and/or other compounds having an aldehyde functional group as the active ingredient to combat the growth of bacteria. After the addition of a predetermined amount of biocide over a prescribed time period (commonly referred to as a “slug”), a water sample is collected manually at various downstream sampling points and the samples are taken to a laboratory where any of a number of known analytical methods can be used to detect the presence and determine the concentration of any biocide in the sample of injection water. Once the samples have been received, the laboratory generally requires several hours to report the concentration of any biocide present in the water system. This practice is followed on a regular basis and after the addition of biocide into seawater at the point of water intake. This method of analysis is time-consuming and is not always practical at remote locations along the pipeline. Due to the complexity of some water injection networks in large oil fields, including those comprised of remote locations, the water distribution system cannot be effectively monitored by personnel at the sites for treatment and measurement of residual biocide concentration.

Additionally, due to the high volumetric flow of water and pipeline length, it is often difficult to precisely determine when the biocide slug will arrive at the water sampling point, leading to a missed opportunity to measure the biocide concentration.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method for measuring biocide concentration in biocide treated seawater in an oilfield pipeline. The method includes measuring pH of the biocide treated seawater in the oilfield pipeline. When the pH of the biocide treated seawater is below 6.8, a plurality of samples are collected using an autosampler. Then the method includes correlating the pH of the biocide treated seawater with the biocide concentration and stopping collection of the plurality of samples when the pH is 7.8 or higher.

In another aspect, embodiments disclosed herein relate to a system for measuring biocide concentration in a seawater in an oilfield pipeline. The system includes a seawater plant, a surge tank located downstream from the seawater plant, a water supply plant located downstream from the surge tank, a second seawater sampling location on the oilfield pipeline immediately downstream from the water supply plant, a water injection plant located upstream from a plurality of injection wells, a plurality of seawater sampling locations on the oilfield pipeline, each of the plurality of seawater sampling locations located immediately upstream from each of the plurality of water injection wells, and a plurality of water injection wells configured to inject the seawater into a plurality of hydrocarbon-bearing reservoirs. The first, second, and the plurality of seawater sampling locations include a pH monitoring system and an autosampler. The autosampler is used to collect a plurality of samples of the biocide treated seawater from the oilfield pipeline.

Other aspects and advantages of the claimed subject matter will be apparent from the following description and the appended claims.

### BRIEF DESCRIPTION OF DRAWINGS

FIG. 1 is a schematic diagram of a biocide concentration measuring system in accordance with one or more embodiments.

FIG. 2A is a pictorial representation of a pH monitoring system to measure the pH of the biocide treated seawater in one or more embodiments of the present disclosure.

FIG. 2B is a pictorial representation of an autosampler in accordance with one or more embodiments.

FIG. 3 is a graphical representation of the measured pH as a function of biocide concentration in one or more embodiments of the present disclosure.

FIG. 4 is a graphical representation of the measured pH as a function of biocide concentration in one or more embodiments of the present disclosure.

### DETAILED DESCRIPTION

Seawater may be injected to maintain reservoir pressure and increase the efficiency of oil production. The distribution pipelines normally form a grid to supply water to a number of injection wells in the vicinity of the production wells. Prior to use, seawater may be treated with a biocide to protect piping from microbial induced corrosion. Because of the overall length of the pipeline system, a drop in the effective concentration of biocide can occur at the point of use. The reduction in biocide concentration is due to the degradation of the active ingredient(s) present in the biocide formulation. Hence, it is important to know the actual concentration of biocide present in water at the point of use.

The concentration of biocide in seawater is conventionally determined by manually collecting samples at various downstream sampling points and the samples are taken to a laboratory where any number of analytical methods may be used to detect the presence and determine the concentration of any biocide in the sample of injection water. This process is time consuming and can result in a lag time between determining a concentration of biocide a pipeline segment and being able to adjust the concentration in a desired timeframe. Thus, one or more embodiments of the present disclosure relate to a system and method to monitor the biocide concentration in a stream of injection seawater on a continuous basis. Another aspect of the disclosure is to determine the concentration of biocide in the water system at the point of use, and at interim sampling points in real-time, utilizing means capable of determining the presence of biocide.

In one aspect, embodiments disclosed herein relate to a system and method for the automated sampling of biocide treated seawater from an oilfield pipeline to measure the biocide concentration. The sampling is automated by measuring the pH of the biocide treated seawater to efficiently and effectively monitor the biocide concentration in the seawater. The pH of the biocide treated seawater may be correlated to the concentration of biocide in the biocide treated seawater system at the point of use and at interim sampling points in real-time.

The system in accordance with the present disclosure is configured and operated to monitor for the presence of a biocide in seawater with minimal human intervention. The automated system is based on measuring the pH of the seawater. The systems and methods described herein may be used to measure any type of biocide. Biocides used to treat seawater may include but are not limited to tetrakis(hydroxymethyl)phosphonium sulfate (THPS), glutaraldehyde (GLUT), benzalkonium chloride (BAC), and combinations thereof.

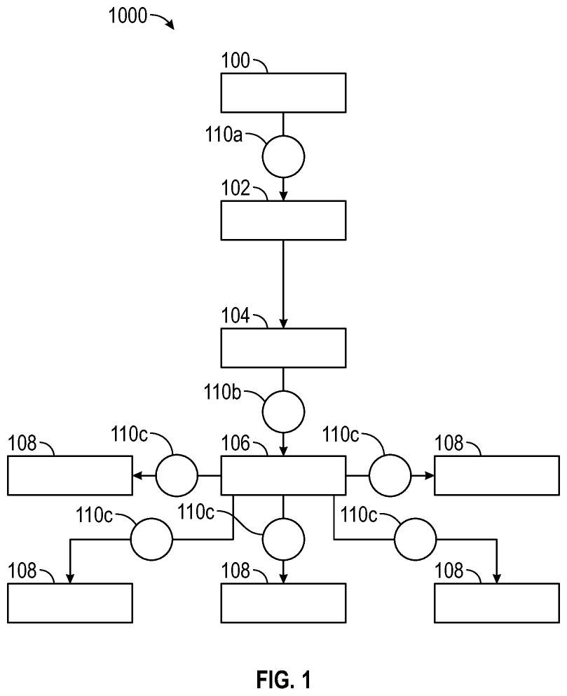

FIG. 1 shows a schematic representation of an automated system for measuring biocide concentration in biocide treated seawater in accordance with one or more embodiments of the present disclosure. The system 1000 includes a seawater plant 100, a surge tank 102, a water supply plant 104, a water injection plant 106, and a plurality of water injection wells 108. These components are fluidly connected by oilfield pipeline, which is indicated by arrows in FIG. 1. The oilfield pipeline is configured to transport seawater from the seawater plant 100 to the injection wells 108. The system 1000 also includes a plurality of seawater sampling locations 110 located along the pipeline. The components in the system 1000 are connected pipelines which carry the biocide treated seawater throughout the system. Each seawater sampling location may include a pH monitoring system and an autosampler.

In accordance with one or more embodiments of the present disclosure, seawater may be collected from a source of seawater and transferred to a seawater plant 100. The collected seawater may be treated with one or more biocides in a seawater plant 100 to produce biocide treated seawater. Prior to treating the seawater with the biocide, the liquid biocide may be stored at a tank farm area. As is common practice in the art, biocide may be injected into the seawater plant 100 at weekly intervals at specified dosing points. The dosing points may be located at the plant outlets to protect the piping network from corrosion. The concentration of biocide in the seawater at the seawater plant 100 may be in an amount ranging from about 500 ppm to about 1000 ppm.

Generally, the pH of seawater may be from about 7 to about 8. The pH of the seawater is expected to decrease after addition of the acidic biocide. The seawater flow rate may be in a rate ranging from about 6.0 to about 8.0 MMBD (Million Barrels Per Day). The high flow rate of the seawater into the seawater plant 100 dilutes the biocide and consequently also raises the pH. Thus, the corresponding pH of the biocide treated seawater may be in the range from about 5.0 to about 6.5 at the dosing point and may have minimal negative impact on the piping such as acid induced corrosion. The pH of the treated seawater may change over time and at different locations along the pipeline because the pH is related to the biocide concentration. The biocide concentration changes over time as the components of the biocide degrade, thus resulting in a change in pH. A pH of less than 6.8 indicates a high biocide concentration. On the other hand, a pH of 7.8 or more indicates a low or undetectable biocide concentration. The seawater sampling locations 110 are configured to monitor the pH of the seawater using a pH monitoring system to correlate the pH to a given biocide concentration at various locations in the system 1000.

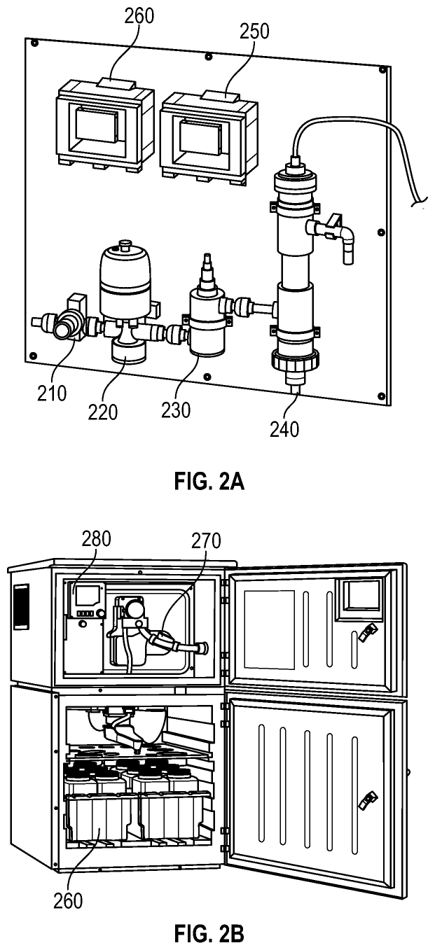

In one or more embodiments, the pH of the biocide treated seawater is measured by a pH monitoring system, which is a part of a plurality of sampling locations 110. FIG. 2A shows a pictorial representation of a pH monitoring system used to measure the pH of the biocide treated seawater. A preliminary test sample of biocide treated seawater is collected from the sampling location on the pipeline and is transferred to the pH monitoring system via an inlet 210 which is connected to the pipeline. The preliminary test sample of biocide treated seawater is transferred to a pH analyzer 220 to measure the pH. The measured pH is displayed on a screen 250, which is connected physically to the pH monitoring system. The pH of the preliminary test sample will determine if the autosampler, which may be located at a plurality of sampling locations 110, will collect a sample to measure biocide concentration. If the pH of the preliminary test sample is in the previously specified range, the seawater will be collected from the pipeline by the autosampler according to operator needs. For example, a sample may be collected by the autosampler every 10 minutes. If the pH is not within the specified range for sample collection, the pH will continue to be monitored until it reaches the threshold pH. [The pH analyzer 220 is connected to a backup pH analyzer 230 which is connected to a secured sampler box 240. The secured sampler box includes a fluid outlet and is used for cleaning purposes and air release in the pH monitoring system, and is unrelated to other sampling activities. The pH monitoring system also includes a backup screen 260, which may be used for other monitoring, like conductivity measurements, which can be a secondary function of the backup pH analyzer. The backup pH analyzer may be used during maintenance activities. The electrical source for the pH analyzer may be a battery or any other electrical source available on site.

As shown in FIG. 1, a first seawater sampling location 110a is located on the oilfield pipeline downstream from the seawater plant 100 and upstream from the surge tank 102. Each sampling location 110 includes a pH analyzer to measure pH and an autosampler to collect seawater samples. The seawater sampling location 110a may be located at any position along the pipeline in between the seawater plant 100 and the surge tank 102. For example, the sampling location 110a may be located immediately downstream from the seawater plant to as to obtain data about the seawater as it exits the seawater treatment plant 100. Additionally, any number of sampling locations 110 may be on the pipeline between the seawater plant 100 and the surge tank 102. The number and position of sampling locations 110 may be adjusted based on the length of the pipeline and the amount of seawater flow, for example.

Typically, the seawater exiting the seawater plant 100 has a high concentration of biocide, as it was just treated, and therefore a correspondingly low pH. The pH is monitored using an online pH analyzer at the sampling location 110a to determine when samples should be collected by the autosampler. To calibrate and maintain the system, the pH of the seawater may be measured manually. The autosampler may start automatically collecting samples of the biocide treated seawater when the pH of the biocide treated seawater is lower than 6.8. The autosampler may be custom designed as needed or may be a commercially available model. An example of a suitable commercial autosampler is an Automatic Water Sampler Liquistation CSF48 available from Endress+Hauser. Biocide treated seawater samples may be collected at any required time interval, such as weekly. A pH of 6.8 or lower indicates that the biocide concentration in the biocide treated seawater is 500 ppm or higher.

In one or more embodiments, the autosampler automatically collects samples of the biocide treated seawater. FIG. 2B is a pictorial representation of the autosampler. used to measure the pH of the biocide treated seawater. An example of a commercially available autosampler suitable for the present disclosure is the Automatic Water Sampler Liquistation CSF48 available from Endress+Hauser, The autosampler includes sampling bottles 260, flow tubes 270, a sample inlet (not shown), a valve inlet for manual pH verification (not shown), a power source (not shown), and program panel 280. The program panel 280 may be used to specify an autosampler collection rate, for example, it may be set to collect a sample every 10 minutes. The volume of seawater collected is in the range of between 50 mL to about 300 mL per sample. The volume of sample collected may have a lower limit of any of 50 mL, 60 mL, 70 mL, 80 mL, 90 mL, 100 mL, 110 mL, 120 mL, 130 mL, 140 mL, 150 mL, 160 mL, 170 mL, 180 mL, 190 mL, 200 mL, 210 mL, 220 mL, 230 mL, and 240 mL and an upper limit of any of 250 mL, 260 mL, 270 mL, 280 mL, 290 mL, and 300 mL where any lower limit may be paired to any mathematically compatible upper limit.

Samples of biocide treated seawater may be collected by the autosampler until the pH is 7.8 or higher. A pH of 7.8 or higher indicates that the biocide concentrated in the biocide treated seawater is less than 100 ppm. An additional dosage of biocide may be added to lower the pH at the seawater plant 100. Samples may be further collected at sampling locations 110 to measure the pH again.

The seawater from the seawater plant 100 is fed into a surge tank 102 which is located downstream from the seawater plant 100. The surge tank 102 controls pressure variation caused by the rapid changes in the velocity of the biocide treated seater from the seawater plant 100.

From the surge tank, the biocide treated seawater is transferred to a water supply plant 104 which is located downstream from the surge tank 102. The water supply plant 104 is configured to increase the pressure of the treated seawater stream enabling the treated seawater to reach the water injection wells 108. In one or more embodiments, additional biocide may be added at the water supply plant 104 if the concentration of biocide is too low.

A second seawater sampling location 110b is located on the oilfield pipeline immediately downstream from the water supply plant 104 and upstream from the water injection plant 106. The seawater sampling location 110b may be located at any position along the pipeline in between the water supply plant 104 and the water injection plant 106. Additionally, any number of sampling locations 110 may be on the pipeline between the water supply plant 104 and the water injection plant 106. The number and position of sampling locations 110 may be adjusted based on the length of the pipeline and the amount of seawater flow, for example.

The pH is monitored at the sampling location 110b as described above. Samples are collected using the autosampler also as described above. Biocide may be added based on monitoring of the pH at the seawater plant 100 as described above.

The water injection plant 106 supplies water to a plurality of water injection wells 108. A third seawater sampling location 110c is located on the oilfield pipeline immediately downstream from the water injection plant 106 and upstream from the water injection wells 108. The seawater sampling location 110c may be located at any position along the pipeline in between the water injection plant 106 and water injection wells 108. Any number of sampling locations 110 may be on the pipeline between the water injection plant 106 and the water injection wells 108. The pH is monitored and samples are collected using the autosampler as described above.

Finally, the oilfield pipeline upstream of each water injection well 108 includes a seawater sampling location 110c. As such, the seawater may also be monitored and collected immediately before being injected into a well. The pH monitoring and sample collection are conducted as described above.

Once samples are collected using the autosampler at each of the aforementioned sampling locations, the biocide concentration may be measured. The biocide concentration may be measured by any appropriate method such as chromatography or titration methods. For example, and as is understood by those skilled in the art, a plurality of standards with known biocide concentration may be prepared and their response measured by any appropriate technique. An example of a suitable titration method is iodometric titration. The choice of titration method may depend on the type of biocide used to treat the seawater.

The concentration of biocide treated seawater samples collected from sampling locations may be subsequently determined by measuring their response by any appropriate technique. The concentration of biocide in the biocide treated seawater may be used to determine the amount of biocide that added to the sea water in the seawater plant 100. If the concentration of biocide in the biocide treated seawater is less than 500 ppm, additional biocide may be added to the system to adjust the concentration of biocide. In contrast, if the concentration of biocide is greater than 1000 ppm, the next dose will be determined accordingly.

### EXAMPLES

The following examples are merely illustrative and should not be interpreted as limiting the scope of the present disclosure.

Biocide treated seawater samples were collected from various sampling locations. The pH of the biocide treated seawater samples was measured.

Table 1 shows the pH measured from different pipelines at the Uthmaniah Water Supply Plant (UWSP). Pipelines-1, pipeline-2, pipeline-3, and pipeline-4 correspond to pipelines at the water supply plant, such as the pipeline shown as 104 in FIG. 1. The pH of collected samples was measured in the laboratory using a commercially available pH meter.

TABLE 1

| Sample | Pipeline 1 | Pipeline 2 | Pipeline 3 |
|---|---|---|---|
| 1 | 7.90 | 7.90 | 7.60 |
| 2 | 7.70 | 7.90 | 7.60 |
| 3 | 6.85 | 7.90 | 7.60 |
| 4 | 6.55 | 7.90 | 7.60 |
| 5 | 6.30 | 7.90 | 7.60 |
| 6 | 7.05 | 7.90 | 7.60 |
| 7 | 7.60 | 7.90 | 7.30 |
| 8 | 7.60 | 7.90 | 7.20 |
| 9 | 7.70 | 7.50 | 7.35 |
| 10 | 7.70 | 7.40 | 7.45 |
| 11 | 7.75 | 7.30 | 7.60 |
| 12 | 7.75 | 7.30 | 7.60 |
| 13 | 7.75 | 7.40 | 7.65 |
| 14 | 7.75 | 6.85 | 7.65 |
| 15 | 7.75 | 6.40 | 7.65 |
| 16 | 7.75 | 6.90 | 7.65 |
| 17 | 7.75 | 7.70 | 7.65 |
| 18 | 7.75 | 7.90 | 7.65 |
| 19 | 7.75 | 7.90 | 7.65 |
| 20 | 7.75 | 7.90 | 7.65 |
| 21 | 7.75 | 7.90 | 7.65 |
| 22 | 7.75 | 7.90 | 7.65 |
| 23 | 7.75 | 7.90 | 7.65 |
| 24 | 7.75 | 7.90 | 7.65 |

Table 2 shows the biocide concentration measured from several automatically collected samples from pipelines 1 and 2 as shown in Table 1. As seen below, a pH of 6.8 or lower corresponds to a biocide concentration of greater than 500 ppm.

TABLE 2

| pH | Biocide concentration (ppm) | Pipeline |
|---|---|---|
| 6.85 | 1279 | Pipeline 1 |
| 6.55 | 801 | Pipeline 1 |
| 6.30 | 1402 | Pipeline 1 |
| 7.05 | 155 | Pipeline 1 |
| 7.60 | 0.05 | Pipeline 1 |
| 7.30 | 110 | Pipeline 2 |
| 7.40 | 54 | Pipeline 2 |
| 6.85 | 511 | Pipeline 2 |
| 6.40 | 801 | Pipeline 2 |
| 6.90 | 199 | Pipeline 2 |

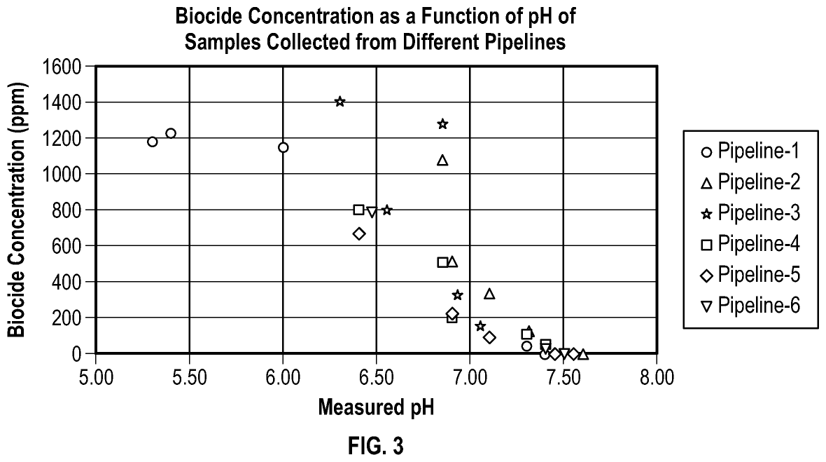

FIG. 3 shows the biocide concentration as a function of pH measured from a plurality of pipelines. As shown, a pH of 6.8 or lower corresponds to a biocide concentration of 500 ppm or higher, a pH of between 6.8 and 7.7 corresponds to a detectable biocide concentration of between 0.05 and up to 500 ppm, and a pH of higher than 7.7 corresponds to an undetectable biocide concentration.

An iodometric titration was used to determine the biocide concentration. 50 mL of biocide seawater sample was added to 4 mL of disodium phosphate and 2 mL of vinyl benzene sulfuric acid sodium salt. The solution was then titrated with a 0.025 N iodine solution. The biocide is a tetrakis(hydroxymethyl) phosphonium sulfate (THPS) type which has a detection limit of 0.05 ppm.

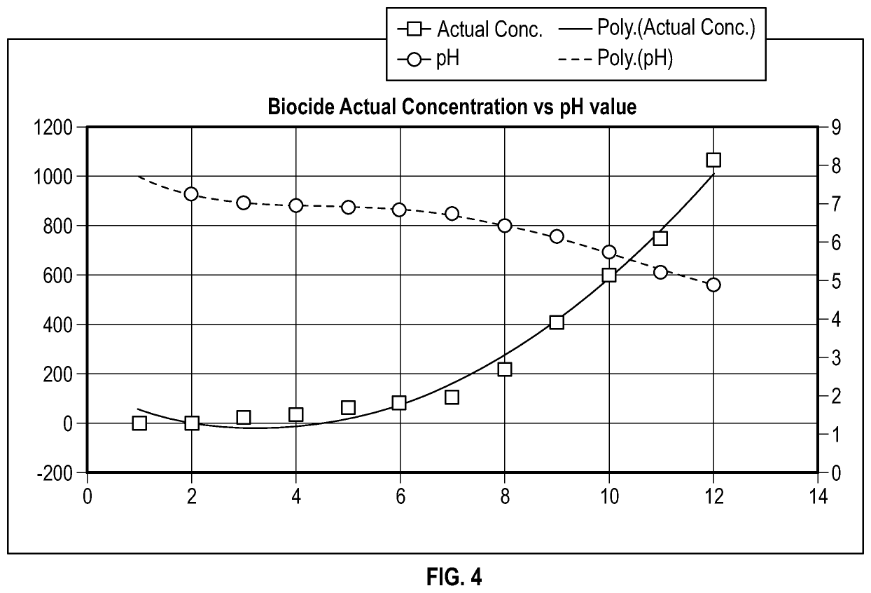

FIG. 4 shows that the relationship between the biocide concentration prepared in the lab from neat chemicals is an inverse relationship with respect to the pH values for the measured samples.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method for measuring biocide concentration in biocide treated seawater in an oilfield pipeline, the method comprising:
measuring pH of the biocide treated seawater in the oilfield pipeline;
when the pH of the biocide treated seawater is below 6.8, collecting a plurality of samples using an autosampler;
correlating the pH of the biocide treated seawater with the biocide concentration; and
stopping collection of the plurality of samples when the pH is 7.8 or higher.

2. The method of claim 1, wherein the biocide is an acidic biocide.

3. The method of claim 2, wherein the acidic biocide is tetra(hydroxy methyl) phosphonium sulfate or glutaraldehyde.

4. The method of claim 1, wherein the autosampler comprises a plurality of sampling bottles, a plurality of flow tubes, a sample inlet, a valve inlet, a power source, and a program panel.

5. The method of claim 1, further comprising, before measuring pH of the biocide treated seawater, adding the biocide to seawater to produce the biocide treated seawater.

6. The method of claim 1, wherein when the pH of the biocide treated seawater is less than 6.8, the biocide concentration is at least 1000 ppm.

7. The method of claim 1, wherein when the pH of the biocide treated seawater is greater than 7.8, the biocide concentration is 100 ppm or less.

8. A system for measuring biocide concentration in a seawater in an oilfield pipeline, comprising:
a seawater plant configured to treat the seawater with biocide to produce biocide treated seawater;
a first seawater sampling location on the oilfield pipeline immediately downstream from the seawater plant;
a surge tank located downstream from the seawater plant configured to control pressure variation caused by rapid changes in biocide treated seawater velocity from the seawater plant;
a water supply plant located downstream from the surge tank configured to store the biocide treated seawater prior to transferring into a water injection plant;
a second seawater sampling location on the oilfield pipeline immediately downstream from the water supply plant;
the water injection plant located upstream from a plurality of injection wells and configured to distribute water from the water supply plant into the plurality of water injection wells; and
a plurality of seawater sampling locations on the oilfield pipeline, each of the plurality of seawater sampling locations located immediately upstream from each of the plurality of water injection wells and comprising an autosampler, the plurality of water injection wells configured to inject the seawater into a plurality of hydrocarbon-bearing reservoirs;
wherein each of the first, second, and the plurality of seawater sampling locations comprises a pH monitoring system and an autosampler, and
wherein the autosampler is configured to collect a plurality of samples of the biocide treated seawater from the oilfield pipeline.

9. The system of claim 8, wherein the autosampler comprises a plurality of sampling bottles, a plurality of flow tubes, a sample inlet, a valve inlet, a power source, and a program panel.

10. The system of claim 8, wherein the pH monitoring system comprises an inlet, a pH analyzer, a backup pH analyzer, a secured sampler box, a screen, and a backup screen.

11. The system of claim 8, wherein the autosampler is configured to collect samples when the pH of the biocide treated seawater is less than 6.8.

12. The system of claim 11, wherein the autosampler is configured to collect samples until the pH of the biocide treated seawater is greater than 7.8.
